(19) United States
(12) Patent Application Publication
(10) Pub. No.: US 2005/0010284 A1
(43) Pub. Date: Jan. 13, 2005

(54) Method for decreasing bioprosthetic implant failure

(76) Inventor: Kenton W. Gregory, Portland, OR (US)

Correspondence Address: MARGER JOHNSON & MCCOLLOM PC, 1030 SW MORRISON STREET, PORTLAND, OR, 97205, US

(21) Appl. No.: 10/888,457

(22) Filed: Jul. 9, 2004

Related U.S. Application Data
(60) Provisional application No. 60/486,661, filed on Jul. 10, 2003.

(51) Int. Cl. A61F 2/24; A61F 2/02

(52) U.S. Cl. 623/2.13; 623/2.42; 623/920

(57) ABSTRACT

Thus, a method is provided for substantially decreasing the failure of a bioprosthetic implant in a human being. The method comprises providing a lipid lowering medication. Then, the human being is treated with the lipid lowering medication to substantially decrease the bioprosthetic implant failure and substantially lowering the need for bioprosthetic implant replacement. Preferably, the lipid lowering medication is a statin, and more preferably the lipid lowering medication is a HMG CoA reductive inhibitor (3-hydroxy-3 methyl-glutamyl coenzyme A reductase inhibitor).

### BACKGROUND OF THE INVENTION

This invention relates to a method for substantially decreasing the failure of a bioprosthetic implant. In turn, it can also significantly increase the survival rate of the person who has had the bioprosthetic implant.

Calcific aortic valve stenosis is the most common cause of aortic stenosis. Diabetes Mellitus has been identified as a risk factor for aortic stenosis and published case reports associate hypercholesterolemia with aortic stenosis as well. Symptomatic patients have a mortality rate of 25% at one year and 50% at two years. Aortic valve replacement surgery is currently the only treatment option in symptomatic patients. Presently mechanical and biologic tissue valves are used for valve replacement, as valve repair has not been an option for calcific aortic stenosis.

Bioprosthetic heart valves have an advantage over mechanical heart valves, as they have better hemodynamic profiles, but more important to patients, a reduced need for chronic anticoagulation. The single largest disadvantage of biologic valves is the incidence of bioprosthetic valve degeneration and calcification, leading to valve malfunction and the need for replacement of same.

Calcification and failure of bioprosthetic implants, especially heart valves, is a repeated occurrence and is associated with frequent mortality and morbidity as well as large healthcare costs. To date current treatments have not been effective in preventing heart valve calcification. A significant portion of the pathophysiology of bioprosthetic heart valve failure is associated with implant calcification.

The need for re-operation can be in as short as 4-5 years. There is a 20% failure rate at 10 years and 50% failure rate at 15 years. Almost all bioprosthetic valves degenerate in patients less than 50 years old. The largest problem with re-operation lies in the mortality rate of 10-15% compared to a primary operative mortality rate of 1-2% with the initial implant.

There are no reported post implant therapies known to prevent bioprosthetic calcification or failure. Preventative therapies currently are directed at treatments of the bioprosthesis prior to implant such as Ethanol incubation, reduction of Gluteraldehyde fixatives or AlCl₃ treatments.

### SUMMARY OF THE INVENTION

It is believed that a key means for reducing or eliminating bioprosthetic valve calcification and failure is an understanding of the biological processes leading to valve degeneration. An approach is to undertake a comparison of the histopathology and clinical aspects of calcific aortic stenosis, prosthetic valve degeneration and atherosclerotic coronary artery disease show many similarities. Diabetes and hypercholesterolemia are risk factors.

Implant calcification appears in part to be preceded by adsorption of lipids. Lipid adsorption or absorption may be a key event in initiating the pathologic process. Lipid adsorption may lead to secondary calcification of lipid-bound matrix proteins.

Thus, a method is provided for substantially decreasing the failure of a bioprosthetic implant in a human being. The method comprises providing a lipid lowering medication. Then, the human being is treated with the lipid lowering medication to substantially decrease the bioprosthetic implant failure and substantially lowering the need for bioprosthetic implant replacement. Preferably, the lipid lowering medication is a statin, and more preferably the lipid lowering medication is a HMG CoA reductive inhibitor (3-hydroxy-3 methyl-glutamyl coenzyme A reductase inhibitor). The bioprosthetic implant preferably comprises a heart valve. More preferably, the bioprosthetic implant comprises a mitral or aortic valve. More preferably, the bioprosthetic implant comprises one of a porcine valve and a pericardial valve.

The reducing of the bioprosthetic implant failure is preferably facilitated by reducing calcification of the bioprosthetic implant. Typically, the human being who has undergone the treatment has a higher survival rate. Preferably, the higher survival rate is facilitated by a reduction in the rate of re-installation of a replacement bioprosthetic implant. Moreover, bioprosthetic implant failure can be further decreased by reducing the structural deterioration of the bioprosthetic implant.

### BRIEF DESCRIPTION OF THE DRAWINGS

FIG. 1 is a schematic graphical representation of freedom from SVD who had implanted aortic valves v. number of years of implant surgery for “Never” and “Ever” patients.

FIG. 2 is a schematic graphical representation of % Survival v. number of years of implant surgery for “Never” and “Ever” patients.

FIG. 3 is a schematic graphical representation of % Survival v. number of years of implant surgery for “Never” and “Ever” patients without concomitant CABG.

FIG. 4 is a schematic graphical representation of % Survival v. number of years of implant surgery for “Never” and “Ever” patients with concomitant CABG.

FIG. 5 is a schematic graphical representation of % Survival v. number of years of implant surgery for “Ever” patients with and without concomitant CABG.

FIG. 6 is a schematic graphical representation of % Survival v. number of years of implant surgery for “Never” patients with and without concomitant CABG.

### DETAILED DESCRIPTION OF THE INVENTION

Bioprosthetic implants exhibit physiologic central flow with less gradients than mechanical valves. They are also less thrombogenic and exhibit a reduced need for anficoagulation. However, bioprosthetic valves are less durable. They are prone to tissue calcification and degeneration, especially in younger patients, and require frequent need for higher mortality re-operative replacement.

It has now been ascertained that the prevention of bioprosthetic implant failure can be effected with the treatment of administration of lipid lowering drugs, i.e., statins. The failure of the bioprosthetic implants is due to a great extent to the effects of calcification. The preferred lipid lowering drugs are HMG CoA reductase inhibitors. The prevention of bioprosthetic implant calcification and failure, such as in aortic heart valves, has been hereinafter demonstrated. Furthermore, this treatment should be effective for the reduction of calcification and failure of other bioprosthetic implants such as valves, vascular conduits and non-vascular bioprosthetic implants.

First, the use of lipid-lowering drugs was reviewed since these drugs can prevent atherosclerotic coronary artery disease and cause a regression of existing disease. The most commonly studied and prescribed class of lipid lowering drugs are HMG CoA reductase inhibitors. HMG CoA reductase inhibitors include materials such as Lipitor, Mevacor, Zocor, Pravochol, etc., which reduce hepatic cholesterol synthesis and also cause a secondary increase in hepatic cell LDL receptors (increased clearance of LDL). These processes result in lower plasma LDL, reduce MI, death from MI, progression of ASVD, and reduction of incidents' of stroke. HMG CoA Reductase Inhibitors will have a powerful effect on reducing calcification and failure of valve and other implants due to lipid lowering as well as an anti-inflammatory or other effects. Pre-treatment and immediate treatment can be important to prevent lipid adsorption by the implants. A reduction in inflammatory indicies have also been observed.

The valve population at highest risk for calcific degeneration and need for high-risk operation is in bioprosthetic heart valves. 20-50% in 10-15 years and greater than 50% in less than 10 years for patients younger than 50. Thus, this is the patient population that is most likely to demonstrate a benefit. Lowering lipid levels with lipid lowering drugs will lower implant calcification and reduce structural deterioration and valve failure.

Bioprosthetic heart valve (BPV) failure results in substantial patient morbidity due to hemodynamic compromise followed usually by reoperation. BPV failure is usually due to calcific degeneration, which may be preceded by lipid adsorption. The treatment of BPV with lipid lowering drugs comprising statins, such as HMG-CoA reductase inhibitors, which have been used successfully to reduce atherosclerotic lesions, should reduce BPV failure and overcome the need for recurrent valve replacement. The advantage of this new therapy for patients with bioprosthetic implants is less implant failure, less need for replacement of the implant, less chance of dying. These improvements should also result in less long-term cost of treatment.

Patients with bioprosthetic implants, such as aortic bioprosthetic valves, who have ever been treated with lipid lowering drugs, such as HMG CoA reductase inhibitors, have a substantially reduced need for bioprosthetic implant replacement than patients who have never taken lipid-lowering drugs. More specifically, patients with bioprosthetic implants who have ever been treated with lipid lowering drugs have preferably less than about 10%, more preferably less than about 5%, and most preferably about 0%, need for bioprosthetic implant replacement, than patients who have never taken lipid lowering drugs, after at least about 15 years following surgery to insert the biprosthetic implant. This is based to a great extent on the substantial reduction in valve deterioration due to the use of lipid lowering drugs.

Moreover, patients with bioprosthetic implants, such as aortic bioprosthetic valves, who have ever been treated with lipid lowering drugs have a substantially increased survival rate than patients who have never taken lipid lowering drugs. More specifically, patients with bioprosthetic implants who have ever been treated with lipid lowering drugs have an increased survival rate which is preferably at least about 150%, more preferably at least about 200%, and most preferably at least about 250%, greater than patients who have never taken lipid lowering drugs after at least about 15 years following surgery to insert the biprosthetic implant.

Lipid lowering drugs such as statins, particularly HMG CoA reductase inhibitors, have a powerful effect on reducing calcification and failure of valve and other implants due to lipid lowering as well as an anti-inflammatory effect. Pre-treatment and immediate treatment will be important to prevent lipid adsorption by the implants

Records that had been accumulated on the database for all bioprosthetic heart valve implants performed within the Providence Hospital System in the Portland, Oregon metropolitan area were reviewed. The implant failure and survival of patients with bioprosthetic heart valves was compared to determine whether they had been treated with cholesterol lowering therapy or not. The results of this review verified that patients who were treated with the lipid-lowering drug, regardless of age, showed less implant failure requiring re-operation, and in turn an improved survival rate.

### Bioprosthetic Implant Survey

During 1976-1996, 511 porcine valves and during 1991-2002, 1021 pericardial valves were used for isolated aortic valve replacement. Thirty-nine porcine and 286 pericardial valve patients have EVER reported use of lipid lowering drugs. In 355 porcine and 387 pericardial patients NEVER took lipid-lowering drugs.

Study Group

1. Consecutive bioprosthetic mitral and aortic valve implants at St. Vincent Hospital and Oregon Health Science University for 26 years (1976-2002)

2. All post-op patients were sent annual questionnaires
a. Patients who EVER took a lipid lowering drugs. The includes patients who have only taken a minimum quantity of lipid lowering drugs.
b. Patients who NEVER took a lipid lowering drugs.
c. Implant failures and re-operation for valve replacement
d. Survival
e. Concomitant CABG
f. Age (age distribution for “never” and “ever” patients was determined to be statistically equivalent)
   Survival

1. Implant failures and re-operation for valve replacement

2. Bioprosthetic Aortic Valve Implants

3. Bioprosthetic Aortic Valve Failure-Freedom from Explant for Structural Valve Deterioration

4. Patient Survival for Patients with Bioprosthetic Aortic Valve Replacement

5. Survival for Aortic Valve Implants

Bioprosthetic Valve Implants

Porcine Aortic Valves

507 Carpentier-Edwards Standard

1 Carpentier-Edwards Standard (early)

3 Carpentier-Edwards XX

Bovine Pericardial Aortic Valves

1021 CE Perimount

9 CE Perimount Reduced Cuff

Bioprosthetic Valve Implants In Survey

511 porcine valves 1976-1996

−394 patients included in analysis

−117 patients excluded-missing data

1021 bovine pericardial valves 1991-2002

673 patients included in analysis

−348 patients excluded-missing data

Patients with Bioprosthetic Aortic Valve Implants Treated with HMG CoA Reductase Inhibitors had better survival. Patients with bioprosthetic aortic valve implants treated with lipid lowering drugs, i.e., HMG CoA reductase inhibitors, have had no instance of need for valve replacement for valve deterioration. Mitral valve implant patients treated with lipid lowering drugs, i.e., HMG CoA reductase inhibitors, have strong trends for increased survival and none have required valve replacement, however patient numbers are smaller.

The age distribution of EVER and NEVER groups are similar for porcine and pericardial. The mean and maximum follow-up years were 6.5 and 18 for porcine, 2.5 and 10 for pericardial. No BPV failure (structural valve failure requiring re-operation) was observed in porcine valves EVER treated with HMG CoA reductase inhibitors compared to 17 BPV failures in porcine BPV NEVER treated (p=0.039). For porcine, 15 year survival±standard error was 44.2±11.4% (95% CI=21.9, 64.5) for EVER and 10.7±2.3% (95% CI=6.0, 15.8) (p<0.001) for NEVER (p<0.001). For pericardial, 10 year survival was 72.3±6.1% for EVER and 30.4±7.3% for NEVER (p<0.001). By Cox regression, age, COPD, renal failure, concomitant CABG and NEVER were found to be independent risk factors for survival.

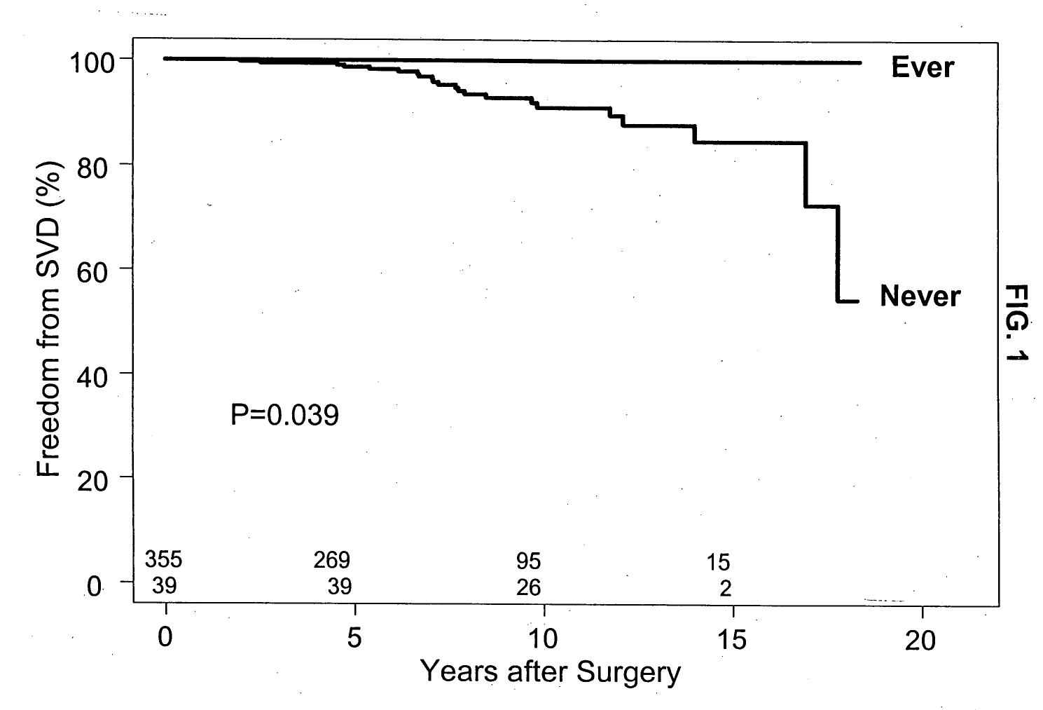

FIG. 1 is a schematic graphical representation of freedom from structural valve degeneration who had implanted aortic valves v. number of years of implant surgery for “Never” and “Ever” patients. There is a statistically significant difference between the two groups for the aortic valve replacement (p-value=0.0395). This clearly demonstrates that patients with bioprosthetic implants, such as aortic bioprosthetic valves, who have ever been treated with lipid lowering drugs, such as HMG CoA reductase inhibitors, have a substantially reduced need for bioprosthetic implant replacement than patients who have never taken lipid lowering drugs.

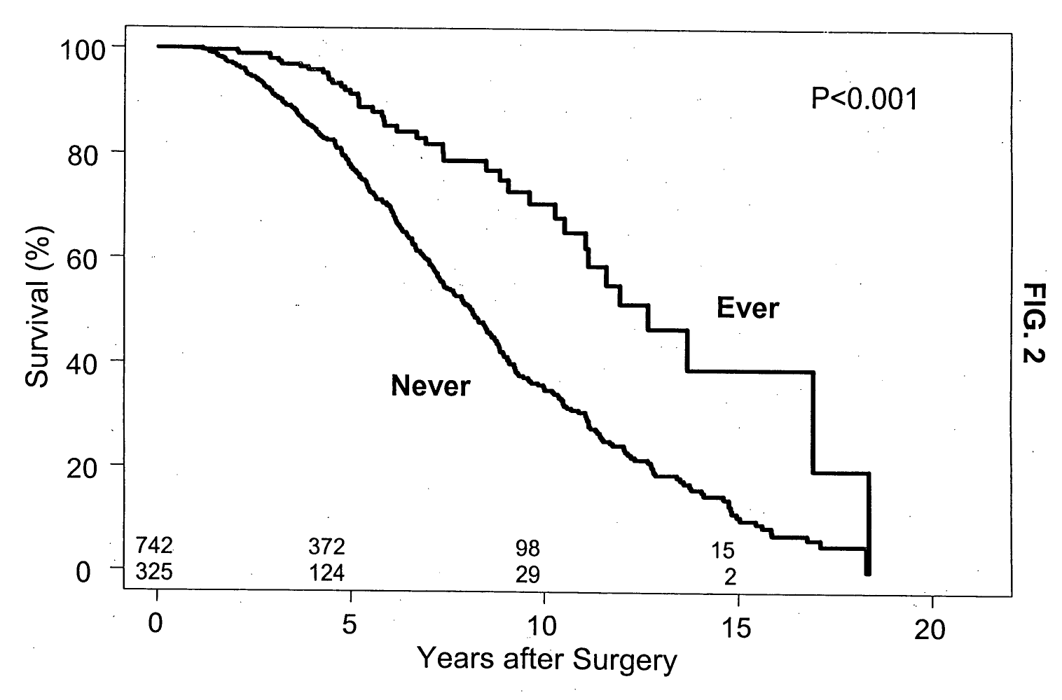

FIG. 2 is a schematic graphical representation of % Survival v. number of years of implant surgery for “Never” and “Ever” patients. There is a statistically significant difference between the two groups for the implant replacement (p-value<0.001). This clearly demonstrates that patients with bioprosthetic implants, such as aortic bioprosthetic valves, who have ever been treated with lipid lowering drugs, such as HMG CoA reductase inhibitors, have a substantially increased survival rate than patients who have never taken lipid-lowering drugs.

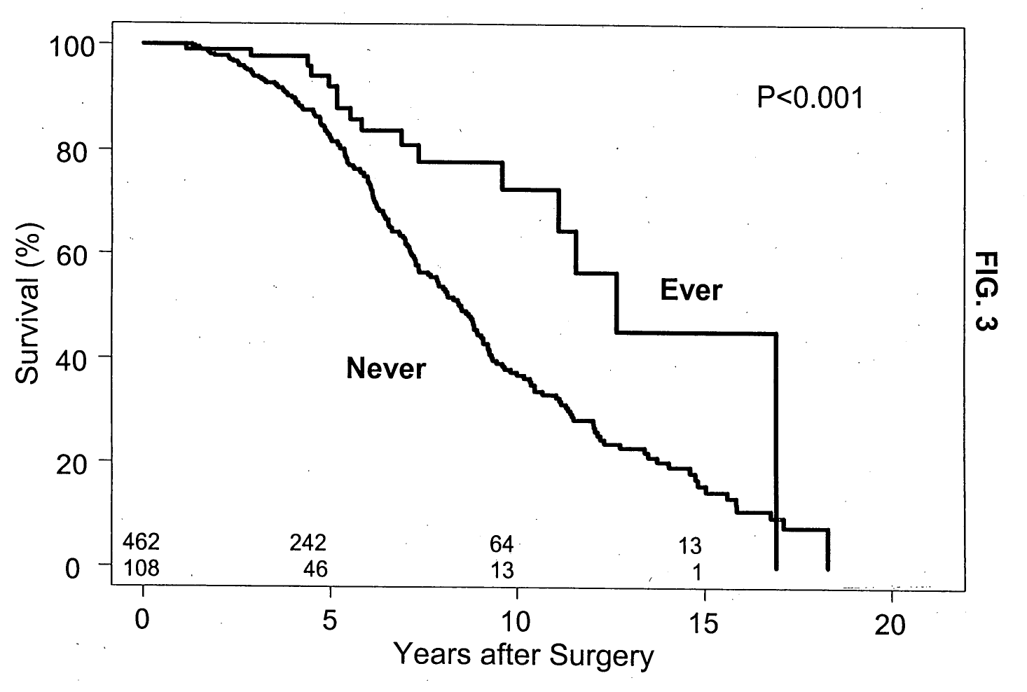

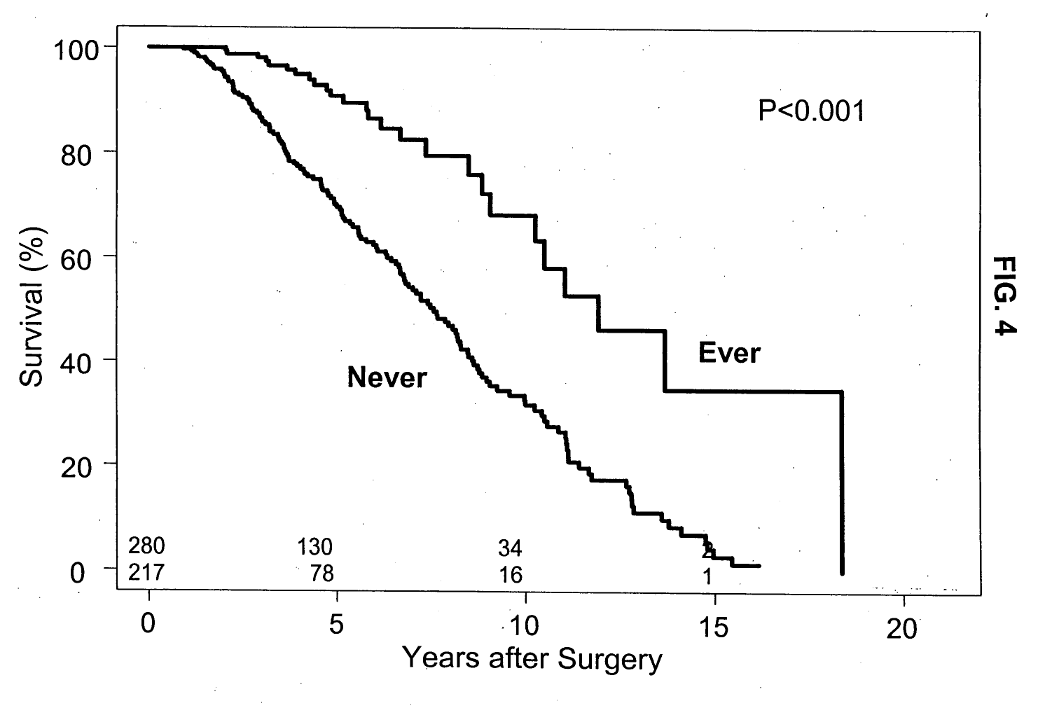

FIG. 3 is a schematic graphical representation of % Survival v. number of years of implant surgery for “Never” and “Ever” patients without concomitant CABG. FIG. 4 is a schematic graphical representation of % Survival v. number of years of implant surgery for “Never” and “Ever” patients with concomitant CABG. There is a statistically significant difference in both FIGS. 3 and 4 between the two groups for the implant replacement (p-value<0.001). This clearly demonstrates that patients with bioprosthetic implants, such as aortic bioprosthetic valves, both with and without concomitant CABG, who have ever been treated with lipid lowering drugs, such as HMG CoA reductase inhibitors, have a substantially increased survival rate than patients who have never taken lipid lowering drugs.

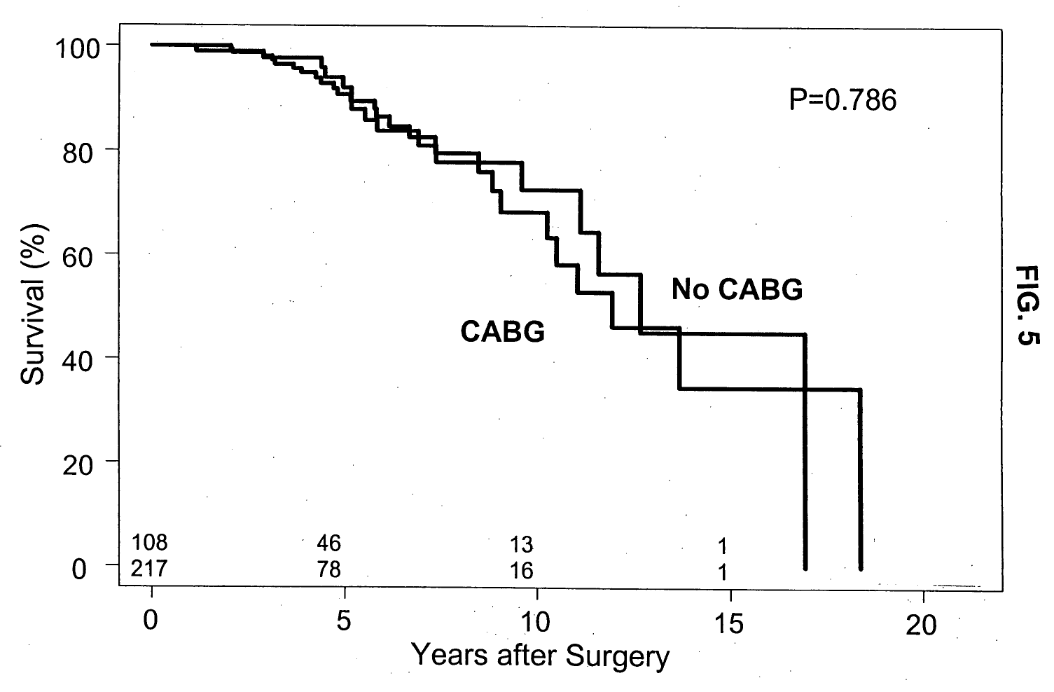

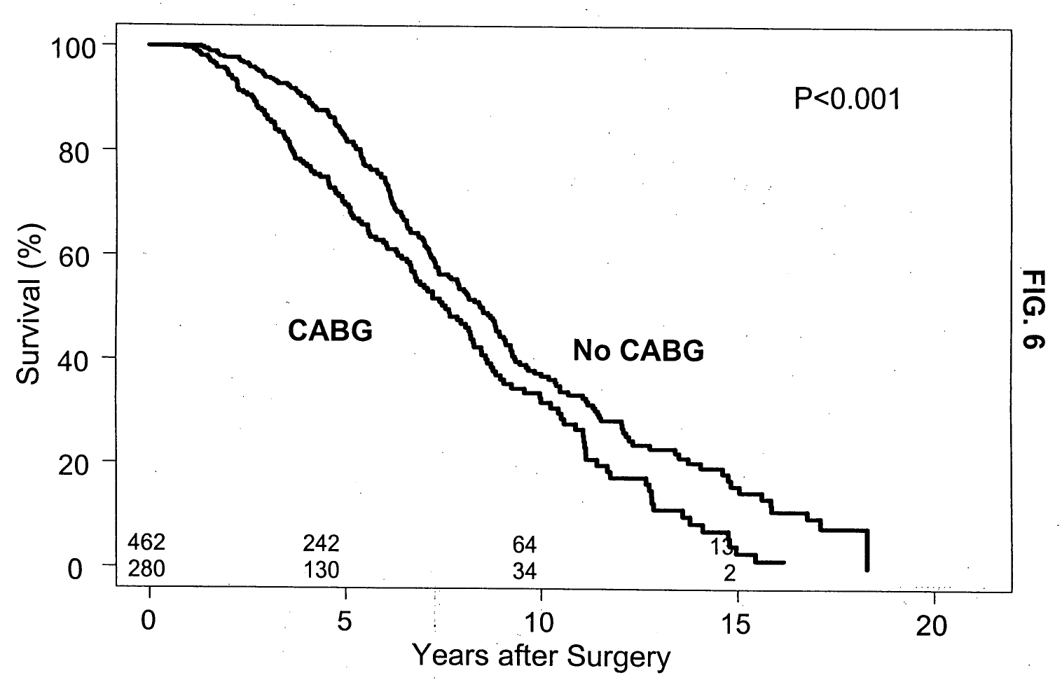

FIG. 5 is a schematic graphical representation of % Survival v. number of years of implant surgery for “Ever” patients with and without concomitant CABG. FIG. 6 is a schematic graphical representation of % Survival v. number of years of implant surgery for “Never” patients with and without concomitant CABG. There is a statistically significant similarities in both FIGS. 5 and 6 between the two groups for the implant replacement (p-value<0.001). This clearly demonstrates that patients with bioprosthetic implants, such as aortic bioprosthetic valves, both with and without concomitant CABG, who have ever been treated with lipid lowering drugs, such as HMG CoA reductase inhibitors, have similar survival rates. This also clearly demonstrates that patients with bioprosthetic implants, such as aortic bioprosthetic valves, both with and without concomitant CABG, who have never been treated with lipid lowering drugs, such as HMG CoA reductase inhibitors, have similar survival rates.

Table 1 below shows the cause of death for “Never” and “Ever” patients without concomitant CABG. Statistically significant improvements in survival rate for patients who died of Valve-related, Cardiac Non-valvular, and Non-cardiac causes is evidenced by the results set forth in Table 1.

| | NEVER | | | EVER | | |
|---|---|---|---|---|---|---|
| | Count | % | %/pat-yr | Count | % | %/pat-yr |
| Valve-related | 75 | 37.9 | 2.9 | 5 | 31.3 | 0.9 |
| Cardiac Non- | 47 | 23.7 | 1.8 | 6 | 37.5 | 1.1 |
| valvular | | | | | | |
| Non-cardiac | 76 | 38.4 | 2.9 | 5 | 31.3 | 0.9 |
| Total | 198 | 100 | 7.6 | 16 | 100 | 2.9 |

1. A method for substantially decreasing the failure of a bioprosthetic implant, comprising:
providing a lipid lowering medication; and
treating a human being having a bioprosthetic implant with said lipid lowering medication to substantially decrease said bioprosthetic implant failure and substantially lowering the need for bioprosthetic implant replacement.

2. The method of claim 1, wherein the lipid lowering medication is a statin.

3. The method of claim 1, wherein the lipid lowering medication is a HMG CoA reductase inhibitor.

4. The method of claim 1, wherein the bioprosthetic implant comprises a heart valve.

5. The method of claim 1, wherein the bioprosthetic implant comprises a mitral valve or an aortic valve.

6. The method of claim 1, wherein the reducing of said bioprosthetic implant failure is facilitated by reducing calcification of said bioprosthetic implant.

7. The method of claim 1, wherein said human being who has undergone said treatment has a higher survival rate.

8. The method of claim 1, wherein bioprosthetic implant failure is decreased by reducing the structural deterioration of said bioprosthetic implant.

9. The method of claim 7, wherein said higher survival rate is facilitated by a reduction in the rate of re-installation of a replacement bioprosthetic implant.

10. The method of claim 1, wherein the bioprosthetic implant comprises one of a porcine valve or a pericardial valve.

11. A method for using a lipid lowering medication for substantially decreasing the failure of a bioprosthetic implant, comprising:
providing said lipid lowering medication; and
treating a human being having a bioprosthetic implant with said lipid lowering medication to substantially decrease said bioprosthetic implant failure and substantially lowering the need for bioprosthetic implant replacement.

12. The method of claim 11, wherein the lipid lowering medication is a statin.

13. The method of claim 11, wherein the lipid lowering medication is a HMG CoA reductase inhibitor.

14. The method of claim 11, wherein the bioprosthetic implant comprises a heart valve.

15. The method of claim 11, wherein the bioprosthetic implant comprises a mitral valve or an aortic valve.

16. The method of claim 11, wherein the reducing of said bioprosthetic implant failure is facilitated by reducing calcification of said bioprosthetic implant.

17. The method of claim 1 1, wherein said human being who has undergone said treatment has a higher survival rate.

18. The method of claim 11, wherein bioprosthetic implant failure is decreased by reducing the structural deterioration of said bioprosthetic implant.

19. The method of claim 17, wherein said higher survival rate is facilitated by a reduction in the rate of re-installation of a replacement bioprosthetic implant.

20. The method of claim 11, wherein the bioprosthetic implant comprises one of a porcine valve or a pericardial valve.
